(19) United States
(12) Patent Application Publication
(10) Pub. No.: US 2004/0161414 A1
(43) Pub. Date: Aug. 19, 2004

(54) Method of curing injured spinal cord and therapeutic agents for that

(76) Inventors: Saburo Kawaguchi, Kyoto-shi (JP); Takeshi Nishio, Kyoto-shi (JP)

Correspondence Address: Finnegan, Henderson Farabow,, Garrett & Dunner, L.L.P., 1300 I Street, N.W., Washington, DC, 20005-3315, US

(21) Appl. No.: 10/777,132

(22) Filed: Feb. 13, 2004

Related U.S. Application Data
(63) Continuation of application No. PCT/JP02/08493, filed on Aug. 23, 2002.

(30) Foreign Application Priority Data
Aug. 23, 2001 (JP) 2001-253586

(51) Int. Cl. A61K 45/00

(52) U.S. Cl. 424/93.7

(57) ABSTRACT

A therapeutic agent for curing injured spinal cord, comprising as the active ingredients glial cells including type-1 and type-2 astrocytes progenitors, and oligodendrocytes progenitors which are CNS glial cells; and a method of curing spinal cord injury by injecting locally an effective dosage of glial cells into the injured spinal cord. It will restore the mobitily of the SCI patients, and thereby will reduce physical and mental burdens of the patients and their family caregiver, and save a heavy burden of medical and social welfare costs.

This is a continuation of an International application No. PCT/JP02/08493 having an international filing date of Aug. 23, 2002.

### BACKGROUND

This invention relates to a novel method to cure spinal cord injuries (SCI) and therapeutic agents for that. In detail, this invention relates to the method to cure spinal cord injuries by local injection of CNS glial cells into injured sites of the spinal cord of SCI patients and therapeutic agents whose active constituent is CNS glial cells.

Spinal cord injuries induce serious symptoms: paraplegia (paralysis of lower extremities on both sides) or quadriplegia or even respiratory paralysis in addition to quadriplegia, and accordingly a wheelchair or a bedridden life is unavoidable. To date effective therapies to cure SCI have not been found yet. Being deprived of the freedom of hand and foot by a momentary accident of traffic or sports the patients desperately hope to move his or her own hands and feet and walk again by restoration of the damaged nerve pathways.

Since the end of the 19th century, it has long been believed that the mammalian CNS pathways do not regenerate at all or regenerate very little if any with no functional significance [For example, Rámon y Cajal, Degeneration and Regeneration in the Nervous System, 1959, Hafner, New York (1928)]. However, studies over the last two decades have revealed that functionally significant regeneration of the mammalian CNS pathways is possible, and accordingly negated the widely-held concept of failure of regeneration.

A hypothesis regarding CNS environment has newly arisen and permeated as a dogma. It claims that environment of the mammalian CNS is non-permissive to axonal growth and consequently it is necessary to make the environment permissive to induce regeneration of axons. Schwab and his collaborators found myelin associated growth inhibitory factor in the CNS white matter and supposed that the factor is responsible for the non-permissiveness of the mammalian CNS. In fact, they reported that neutralization of the factor by antibody induces regeneration of the pyramidal tract after transection and extension of the tract occurs across the lesion in adult rats [Nature, 343(18), 269(1990)]. Besides their reports, various attempts to make the CNS environment permissive were made to induce regeneration of the spinal cord in adult rats: transplantation of a peripheral nerve segment, Schwann cells, olfactory ensheathing cells (OEC, glial cells specific to olfactory nerve and bulb). Cheng and his collaborators reported the occurrence of functional recovery after removal of spinal cord segments in adult rats by bridging a peripheral nerve segment in the gap of the vacated spinal cord [Science, 273 (26), 510 (1996)]. Guest and his collaborators reported the occurrence of regeneration of the spinal cord by grafting Schwann cells, that is, glial cells of the PNS [Exp. Neurol., 148, 502 (1997)]. Li and his collaborators reported the occurrence of regeneration of the pyramidal tract and functional recovery by grafting cultured OEC into the lesion after partial transection of the upper cervical cord [Science, 277(26), 2000 (1997)]. Regenerated projections by these attempts to make environment permissive, however, were small in amount and short in length (at most 10 mm) and mostly aberrant, not reaching the proper targets. Consequently the extent of functional recovery was so small that hindlimbs could not fully support the body weight. To let the SCI patients free from the wheelchair and walk again by their own feet, it is seriously wanted to develop a novel curative method that makes it possible to reconstruct neural projections similar to normal.

The objective of this invention is to provide a novel method that achieves restoration of neural projections similar to normal in amount (the number of projection neurons), length (extension of axons), path and termination, and enables the SCI patients to walk again. It is also to provide therapeutic agents for the method. It is expected that this invention reduces physical and mental burdens of the patients and their family caregiver, and saves a heavy burden of medical and social welfare costs.

### SUMMARY OF THE INVENTION

This invention is based on our hypothesis that the local conditions in the lesion but not the global non-permissive environment of the CNS are responsible for the failure of axonal regeneration. The hypothesis is quite different from the currently held view but consistent with all findings in our spinal cord injury studies: we found that marked regeneration of transected CNS pathways occurs spontaneously after a sharp cut in rats younger than one month of age. In adult rats ranging from 2 to 3 months old spontaneous regeneration did not occur presumably because CNS tissue is harder compared with young animals and thus transection induces edema in the lesion. However, when grafted with embryonic rat spinal cord tissue into the lesion axonal regeneration similar to normal projections was induced. Therefore, we presumed that the CNS environment is not globally non-permissive and failure of regeneration is due to deterioration of the local conditions, i.e., perturbation of axon guidance cues that enable growing axons to find correct path and targets. It appeared very likely that various attempts to make CNS environment permissive break consistency of axon guidance cues and consequently restrict regeneration of axons in amount and extension resulting in aberrant projections. On the basis of our hypothesis we attempted to ameliorate the local conditions in the lesion by grafting cultured mixed glial cells harvested from neonatal rat spinal cord, expecting restoration of microenvironment in the lesion. The glial cells were grafted into the lesion after a complete transection of the spinal cord at a level of thoracic segments in adult rats. The grafted animals recovered from complete paraplegia to walking nearly normally. Regenerated projections were similar to normal in amount, extension, path, and termination. This invention was achieved by a novel insight into regeneration of the mammalian CNS. Diametrically opposite to the conventional concept that CNS glial cells impede axonal regeneration we succeeded in achieving neural repair of the spinal cord using such cells. The extent of restoration of neural connections and recovery of function were much higher than those in the attempts performed on the basis of the conventional concept.

This invention offers a method of curing spinal cord injury by transplantation of CNS glial cells into the injured spinal cord in humans or other mammals. The cells for transplantation comprise at least a kind of cultured CNS glial cells other than type-1 astrocytes. This invention offers also therapeutic agents appropriate for the method to cure spinal cord injuries.

The therapeutic agents comprise at least one kind of cultured CNS glial cells other than type-1 astrocytes as an active ingredient and can contain any vehicle permitted for medical treatment. The further feature and advantage of this invention will become clear in the following “the best form of implementation of invention.”

### THE BEST MODE FOR CARRYING OUT THE INVENTION

CNS glial cells consist of type-1 and type-2 astrocytes, oligodendrocytes, microglia, and their progenitors. The therapeutic agents of this invention comprise at least one kind of cultured CNS glial cells other than type-1 astrocytes as an active ingredient; they may be mixed cells of more than two kinds of CNS glial cells. The CNS glial cells preferably include at least one of the three kinds of cells, i.e., type-1 astrocyte progenitors, type-2 astrocyte progenitors, and O4 progenitors. The most appropriate therapeutic agents are comprised mainly of type-2 astrocyte progenitors but may contain type-1 astrocytes, type-2 astrocytes, oligodendrocytes, and microglia. In addition they may contain Schwann cells and olfactory ensheathing glia.

The glial cells to be used in this invention are unrestricted in origin; autologous or allogeneic or xenogeneic CNS glial cells can be used. Among them autologous cells and allogeneic cells are preferable. For clinical application autologous cells can be harvested from the injured spinal cord of the patients themselves. Allogeneic cells can be harvested from aborted embryos or corpse of brain/cardiac death. Xenogeneic cells can be harvested from pig, monkey, or some other mammals. Since the CNS is immunologically privileged site transplanted xenogeneic cells can survive when a small amount of immunosuppressant drugs is administered. Resource of the CNS glial cells is preferably embryos, neonates or young animals but can be aged animals. They can be harvested preferably but not necessarily from the spinal cord. Any other part of the CNS, for example, the cerebral cortex, brainstem or the whole brain can be the source of supply. Glial cells derived from embryonic stem cells or neural stem cells can also be the source of supply. Neural stem cells are classified into adult type, embryonic type, and neuroepithelial type. They can be harvested not only from embyos or neonates but also from adults. Glial cells can be prepared from these resources by conventional biotechnologies using stimulant of EGF, bFGF, CNTF, retinoic acid or T3 [(Genes Dev., 10, 3129-3140 (1996), Neuron, 18, 81-93 (1997), J. Neurosci., 18, 3620-3629 (1998)).

Any cell culture technology can be used for the preparation of glial cells. For example, the spinal cord or the cerebral cortex that was extirpated aseptically is treated with proteinase (e.g., trypsin) to make single cells or a small cluster of cells. Subsequently they are seeded on a Petri dish and cultured in a serum contained medium for a certain period of time in a CO₂ incubator. During culture neurons die early and mixed glial cells survive. For cell culture Dulbecco's MEM (DMEM) containing 10-20% of fetal bovine serum, F-10 medium, or RPMI1640 or some other media can be used. Medium is exchanged every 3-4 days. With days astrocytes become dominant because of their vigorous proliferative potential. Oligodendrocytes can be increased by employing serum free medium when necessary. Percoll density gradient method or adhesive difference method is effective in separating oligodendrocytes from astrocytes.

The therapeutic agents in this invention can be prepared appropriate for application by making cell suspension of the cultured glial cells in a culture medium or a suitable buffered solution like PBS. The medium of cell suspension can contain medically permitted additives unless they depress activity of cells. Cell density for application can be ranged 10³-10⁶ cells/μL, preferably 10⁴-10⁵ cells/μL.

The method to cure spinal cord injuries is injection of an effective amount of the suspension into the lesion site. The method is applicable for humans and other mammals, either for partial or complete transection. There is no restriction to the location of injury: any part of the medulla, cervical, thoracic, lumbar, or sacral segments. The method is applicable for respiratory paralysis, quadriplegia, paraplegia irrespective of severity.

The method is applicable best to spinal cord injuries by a fall accident or sports accident but not necessarily to such traumatic accident. It may also be applicable, for example, to a cerebrovascular damage to the pyramidal tract. The treatment can preferably be performed acutely, namely within 24 hours, preferably within 8 hours. However, the treatment may also be performed in a chronic stage, for example, one week, 5 years, or even more than 10 years after injury. The latter is based on the findings that a considerable number of projection neurons survived 3 months after severance of axons (3 months in the rat correspond to more than 10 years in humans). It appears, therefore likely that regeneration of axons is possible provided local conditions in the lesion are ameliorated.

Any method can be employed to administer suspension of the cultured glial cells to the lesion site so far as it is injected safely and unfailingly. For example, the spinal cord is exposed by laminectomy, then, the suspension can be injected intramedullarly using a microsyringe under a surgical microscope. When high resolution MRI images are obtained the cell suspension can be injected without laminectomy as in the technique of lumbar puncture. The number of CNS glial cells to be injected depends on the extent of injury. Usually it ranges 10³-10⁷ cells, preferably 10⁵-10⁷ cells in total for an adult patient. Prior to the injection of cells immunosuppressant drugs such as cyclosporins, tacrolimus (FK505), cyclophosamid, azathioprines, methotrexate, or mizoribin can be administerd. It is indispensable when xenogeneic cells are transplanted.

### EXAMPLES

This invention is explained more concretely in the following examples, which do not limit the range of invention.

### Example 1

### Preparation of Suspension of the Cultured Mixed Glial Cells Harvested from Neonatal Rat Spinal Cord and Analysis of Their Composition

The spinal cord was extirpated aseptically from 1-2 day-old EGFP-transgenic SD rats [EGFP=enhanced green fluorescent protein, SD=Sprague-Dawley strain; refer to FEBS Letter, 407, 313-319, 1997]. It was dissociated into single cells or a small cluster of cells by trypsin and DNase treatment. They were seeded on a Petri dish at a density of 5×10⁵ cells/75 cm² in DMEM medium supplemented with 10% fetal bovine serum, penicillin 100 units/mL, amphotericin B 2.5 μg/mL, streptomycin 100 μg/mL, and cultured under ordinary conditions. The cell density corresponds to materials taken from 3-4 spinal cords per dish. On the 2nd, 6th, and 10th day DMEM medium of the same constituents was added. In 2 weeks the cells reached confluence. They were dissociated with trypsin-EDTA (prepared by GIBCO/BRL, 0.25% trypsin, 1 mM EDTA), reseeded (1 dish→1 dish) and cultured. The medium exchange was performed every 3-4 days. At 3-4 weeks after starting culture the cells were dissociated with trypsin-EDTA and made into suspension of 4-5×10⁴ cells/μL with DMEM culture medium (1 dish gives 50 μL). The cell suspension was administered in Example 2.

Composition of the cultured mixed glial cells was analysed by antigen specific marker molecules and listed in Table 1. The classification described in Neuroglia, Helmut Kettenmann et al., Oxford University Press (1995) was employed.

TABLE 1
Composition of cultured mixed glial cells
harvested from the rat spinal cord

| | expression of antigen | | | | | abundance |
|---|---|---|---|---|---|---|
| type of cell | Vim¹ | Ran2 | A2B5 | O4 | GFAP | ratio(%) |
| glial lineage progenitors | + | + | + | − | − | 5 |
| type-1 astrocyte | + | + | − | − | − | 25 |
| progenitors | | | | | | |
| type-1 astrocytes | − | + | − | − | + | 5 |
| O2A progenitors | + | − | + | − | − | 0 |
| type-2 astrocyte | + | − | + | − | + | 45 |
| progenitors | | | | | | |
| type-2 astrocytes | − | − | + | − | + | 0 |
| O4 progenitors | − | − | + | + | − | 15 |
| radial glia | + | − | − | − | − | 3 |
| progenitors(RC2+) | | | | | | |
| microglia(ED1+) | − | − | − | − | − | 2 |

¹Vim = vimentin

### Example 2

### Injection of Glial Cells into the Lesion of Injured Spinal Cord

The spinal cord of adult SD rats (♀, 2 months old) was sharply transected completely at a lower thoracic segment. The cell suspension prepared in Example 1 was injected into two sites rostral and caudal to transection by a Hamilton microsyringe in amount of 4-5×10⁴ cells (1 μL) in each site. Locomotor performance was assessed using the BBB scale developed by Basso, Beattie, and Bresnahan [J. Neurotrauma 12:1-21 (1995)]. Briefly, the score 0 and 21 indicate locomotion of a paralyzed rat and normal locomotion respectively. The scores 1 through 8 focus on limb movements in rats that cannot stand or support weight. The scores 9 through 13 describe rats that are able to stand and then step with varying degrees of hind-forelimb coordination. The scores 14 through 20 describe rats that are good walkers with progressive improvements in foot placement, toe clearance, tail position, and trunk stability. In the beginning the spinal cord injured rats treated with glial cell injection were completely paraplegic and anuretic, bearing wet and dirty anogenital region. In 3˜4 days after surgery they started to move hindlimbs. In a week the hindlimbs became able to support weight. In 2 weeks walking with hind-forelimb coordination was observed. In 3 weeks they could walk in a manner similar to normal animals. BBB score was elevated from 0 to more than 15. Regenerated axons examined by tracing methods were similar to normal projections in amount, extension, and path. They terminated in normal targets and formed synapses.

### Example 3

### Preparation of Suspension of Cultured Mixed Glial Cells that were Harvested from Injured Spinal Cord in Adult Rats

The spinal cord of EGFP-transgenic adult SD rats of 60-day-old [c.f. FEBS Letter, 407, 313-319, 1997] was transected partially at a lower thoracic segment. The animals were kept for 1 month. One month after surgery, 2-3 segments including the lesion were dissected out aseptically and treated by proteinase to make single cells or small clusters of cells in a manner similar to that described in Example 1. These cells or cell clusters were seeded on a Petri dish (75 cm²) at a density of 5×10⁶ cells (materials taken from one or two rats) per dish and cultured under ordinary conditions in DMEM medium supplemented with 10% fetal bovine serum, penicillin 100 units/mL, amphotericin B 2.5 μg/mL, streptomycin 100 μg/mL. On the 2nd, 6th, and 10th day DMEM medium of the same constituents was added. Thereafter the medium exchange was performed every 3-4 days. Cell proliferation was much slower in the materials derived from adults compared to those derived from neonates and the cells reached confluence in 3-4 weeks. They were dissociated with trypsin-EDTA as stated, reseeded (1 dish→1 dish) and cultured for another 2 weeks. In 5-6 weeks after the culture was started, cells were collected and suspension was prepared in a manner similar to that described in Example 1 at a density of 4-5×10⁴ cells/μL. The suspension of cultured mixed glial cells derived from the injured spinal cord of adult rats was used for the injection into the lesion in Example 4.

### Example 4

### Injection of Cultured Mixed Glial Cells Derived from the Injured Spinal Cord of Adult Rats into the Lesion of Spinal Cord Injury

The spinal cord of adult SD rats (♀, 2 months old) was sharply transected completely at a level of lower thoracic segments. The cell suspension prepared in Example 3 was injected into the lesion in a manner similar to that described in Example 2. Consequently the animals recovered from complete paraplegia to walking as in Example 2.

### Comparative Example 1

### Injection of Cultured Type 1 Astrocytes into the Lesion

Cell suspension of type-i astrocytes derived from the cerebral cortex of neonatal rats were prepared in a manner similar to the preceding research by Wang JJ et al. (Effects of astrocytes implantation into the hemisected adult rat spinal cord. Neuroscience 65, 973-981, 1995). The spinal cord of adult SD rats (♀, 2 months old) was sharply transected completely at a lower thoracic segment and the cell suspension was injected into two sites rostral and caudal to transection by a Hamilton microsyringe in amount of 4-5×10⁴ cells (1 μL) in each site. The procedure was similar to that described in Example 2. In the beginning the rats were completely paraplegic and anuretic, bearing wet and dirty anogenital region as in Example 2, whereas recovery of function was far inferior to Example 2. In a week slight hindlimb movements occurred but weight support never occurred. Assessment of BBB scale was always lower than score 8.

### Comparative Example 2

### Injection of Activated Cultured Macrophages into the Lesion

We prepared cell suspension of macrophages that were cultured and activated by co-culture of a sciatic nerve segment in a manner similar to the preceding research by Schwartz M et al. (Implantation of stimulated homologous macrophages results in partial recovery of paraplegic rats, 1998). The spinal cord of adult SD rats (♀, 2 months old) was sharply transected completely at a lower thoracic segment and the cell suspension was injected into two sites rostral and caudal to transection by a Hamilton microsyringe in amount of 4-5×10⁴ cells (1 μL) in each site. The procedure was similar to that described in Example 2. In the beginning the rats were completely paraplegic and anuretic, bearing wet and dirty anogenital region as in Example 2, whereas recovery of function was far inferior to Example 2. In a week slight hindlimb movements occurred but weight support never occurred. Assessment of BBB scale was always lower than score 8. The results were very similar to those in Comparative Example 2.

### INDUSTRIAL APPLICABILITY

This invention provides a method for curing spinal cord injury by injection of cultured mixed CNS glial cells into the lesion. The invention achieved repairing of neural connections that were hardly distinguishable from normal in amount (the number of projection neurons), length (extension of axons), path and termination of neurons, extension of axons, path, and termination. Recovery occurs rapidly from complete paraplegia to the extent of not only weight support but also walking with hind-forelimb coordination. This breakthrough has hitherto unachieved despite various endeavors to cure spinal cord injury and provides strategies for effective therapy that is absent at the moment. When effective therapy is developed, it will reduce physical and mental burdens of the patients and their family caregiver, and save a heavy burden of medical and social welfare costs.

What is claimed is:

1. A therapeutic agent for curing injured spinal cord, comprising as active ingredients glial cells including mainly cultured type-2 astrocytes progenitors.

2. The therapeutic agent according to claim 1, wherein the glial cells are mixed glial cells derived from a spinal cord.

3. The therapeutic agent according to claim 1, wherein the glial cells are autologous or allogeneic.

4. The therapeutic agent according to claims 1, wherein the glial cells are contained in an amount of 10³-10⁶ cells/μL.

5. A method of curing spinal cord injury in humans or other mammals, comprising the steps of:
(a) transecting sharply the injured spinal cord; and
(b) injecting locally an effective dosage of glial cells into the transection, the glial cells including mainly cultured type-2 astrocytes progenitors.

6. The method according to claim 5, wherein the glial cells are mixed glial cells derived from a spinal cord.

7. The method according to claim 7, wherein the glial cells are autologous or allogeneic.

8. The method according to claim 5, wherein the injured spinal cord is part of an adult human body, and the cultured CNS glial cells are injected in a total number of 10³-10⁷.
